(19) United States
(12) Patent Application Publication
(10) Pub. No.: US 2006/0058697 A1
(43) Pub. Date: Mar. 16, 2006

(54) Portable health check apparatus and expiration analysis service method using the same

(76) Inventors: Kazuhiro Mochizuki, Tokyo (JP); Toshiki Sugawara, Kokubunji (JP); Masataka Shirai, Higashimurayama (JP); Hiroyuki Uchiyama, Musashimurayama (JP); Masaru Kokubo, Hanno (JP)

Correspondence Address: REED SMITH LLP, Suite 1400, 3110 Fairview Park Drive, Falls Church, VA, 22042, US

(21) Appl. No.: 11/068,868

(22) Filed: Mar. 2, 2005

(30) Foreign Application Priority Data
Sep. 13, 2004 (JP) 2004-264801

(51) Int. Cl. A61B 5/08; G08B 1/08

(52) U.S. Cl. 600/532; 128/920; 128/903; 340/539.13

(57) ABSTRACT

A portable health check apparatus incorporating a highly sensitive and compact expiration analysis sensor and an expiration analysis service offered to a specified user at a specified time. Hydrogen is detected using tungsten oxide produced through metalorganic chemical vapor deposition process. The apparatus has a unit for performing personal authentication by using, for example, a voiceprint of a user. The apparatus is also arranged to call up a user at a predetermined time through a cellular phone. Further, a center server receives detection information of an expiration component via a communication circuit, analyzes or statistically processes the detection information, collates a result of the analysis or statistic process with advice information stored in a database and transmits advice information based on a result of the collation from the server to the cellular phone of the user.

### INCORPORATION BY REFERENCE

The present application claims priority from Japanese application JP2004-264801 filed on Sep. 13, 2004, the content of which is hereby incorporated by reference into this application.

### BACKGROUND OF THE INVENTION

The present invention relates to a portable health check apparatus provided with a gas sensor sensitive to a predetermined gas (for example, hydrogen gas) and an expiration (expired-gas) analysis service using the same.

A method of producing a metal oxide for use in a gas sensor has hitherto been disclosed (for example, see Patent Document 1 (JP-A-2001-59160)). Patent Document 1 describes that in the case of tin oxide SnO₂ representing a metal oxide used most widely and frequently for a gas sensor, an electron beam vapor deposition process using oxygen radical in combination is superior to such a process as plasma excitation chemical vapor deposition (PECVD), metalorganic chemical vapor deposition (MOCVD), sputtering or thermal oxidation.

So far, a cellular phone has also been disclosed which is so constructed as to build in a semiconductor gas sensor sensitive to components contained in expiration exhaled from a human body (for example, see Patent Document 2 (JP-A-2000-341375 or its patent family EP1046910A2/A3)).

### SUMMARY OF THE INVENTION

In the cellular phone built in with a semiconductor gas sensor sensitive to components contained in expiration exhaled from a human body as disclosed in Patent Document 2, methylmercaptan responsible for halitosis or ethylene responsible for an odor after drinking is detected from components contained in expiration exhaled during a naturally held conversation by using a sensor made from tin oxide (SnO₂). On the other hand, since around 1999, the relation between analysis of expired gas and diseases has been clarified, which, combined with such a social change that drastically widespread use of cellular phones has proceeded and a possessor of a cellular phone always carries it, has been causing an increasing need for and spread of applying the expiration analysis incorporated in a cellular phone to not only checking halitosis and intoxication degree after drinking but also health care.

Results of study of expiration chemistry teach that hydrogen is included in expiration components exhaled from a patient suffering from, for example, abnormal growth of intestinal anaerobic bacteria or dyspeptic syndrome, acetone is included in expiration components exhaled from a patient suffering from diabetes, ammonia is included in expiration components exhaled from a patient suffering from liver disease and trimethylamine is contained in expiration components exhaled from a patient suffering from kidney disease. The sensor of SnO₂, however, has poor sensitivity to only hydrogen of these kinds of gases and a novel sensor material has been demanded. A first object of this invention is to provide a portable health check apparatus mounted with a sensor array including a compact sensor highly sensitive to hydrogen.

In conducting health care through the use of a cellular phone as described previously, a problem of privacy protection cannot be neglected and personal authentication, not incorporated in the conventional cellular phone built in with the halitosis/alcohol sensor, is needed. A second object of this invention is to provide a portable health check apparatus capable of offering an easy authentication method which does not burden users.

Further, some kinds of the medical checkup require data collected daily at a constant or predetermined time but the expiration analysis is liable to be forgotten if conducted at the discretion of spontaneousness of cellular phone users. Accordingly, a third object of this invention is to provide a portable health check apparatus capable of conducting the expiration analysis periodically.

Furthermore, the halitosis/alcohol sensor can afford to give a medical checkup within the cellular phone but when taking a health care support for a patient suffering from liver/kidney disease who lives usually domestically at home, for instance, there occurs a case in which a decision cannot be made within a personal cellular phone. A fourth object of this invention is to provide an expiration analysis service method applicable to a case in which an expert diagnosis is to be made for the sake of health care.

Furthermore, in case a patient suffering from liver/kidney disease is monitored periodically through diagnosis by a doctor, there is a possibility that the patient being out suddenly falls in bad condition and a close examination is needed urgently. A fifth object of this invention is to provide a support means in case of emergency.

A typical example of the present invention will be described below. More particularly, a portable health check apparatus according to this invention comprises a mobile radio terminal and a gas sensor arranged at least either internally or externally of the mobile radio terminal, the gas sensor being so constructed as to include a hydrogen sensor, whereby on the basis of results of sensing by the gas sensor, information concerning health of human body corresponding to a sense object by the gas sensor is displayed on the mobile radio terminal.

An expiration analysis service method according to the present invention comprises the steps of receiving, at a center server from the portable health check apparatus through the medium of a communication circuit, detection information relating to an expiration component exhaled from a user of a portable health check apparatus and detected by the portable health check apparatus, applying an information process to the detection information at the center server, collating a result of the information process with a plurality of pieces of advice information stored in a database accessible from the center server and transmitting, from the center server, advice information selected from the plural advice information pieces on the basis of a result of the collation directly or indirectly to the portable health check apparatus belonging to the user. The portable health check apparatus satisfies the requirements as above.

Especially, to attain the first object of the invention, a highly sensitive/compact hydrogen sensor having tungsten trioxide (WO₃) highly sensitive to hydrogen selected as its hydrogen detectable material and being formed through MOCVD (metalogranic chemical vapor deposition) is preferably mounted to the portable health check apparatus.

To attain the second object of the invention, a personal authentication measure represented by any one of voiceprint in, for example, “Hello” uttered by the portable health check apparatus user, password input and authentication omissible registration is preferably incorporated in the portable health check apparatus.

To attain the third object of the invention, a contrivance for enabling a person in charge of a portable health check apparatus user or health care of the user to call up the user at a predetermined time through a cellular phone is preferably mounted to the portable health check apparatus.

Preferably, to attain the fourth object of the invention, an expiration analysis service method is provided in which detection information of an expiration component is received at a center server through the medium of a communication circuit of cellular phones, the detection information is analyzed or statistically processed at the server, a result of the analysis or statistic process is collated with advice information pieces stored in a database and advice information based on a result of the collation is transmitted from the server to a cellular phone of the user.

Preferably, to attain the fifth object of the invention, an expiration analysis service method is provided in which when detection information is proven to be urgent, a notice is automatically given to a hospital the user attends usually and besides position information is transmitted automatically, along with the analysis result, from the cellular phone, a doctor in charge in the receiving hospital confirms these pieces of information to either recommend attendance to the hospital or introduce a hospital near the user by annexing a map.

According to the present invention, a portable health check apparatus mounted with a sensor array including a compact sensor highly sensitive to hydrogen can be provided.

In connection with the first object of the invention, a hydrogen sensor formed from WO₃ through MOCVD process and having a catalytic film formed by depositing Pd by 15 nm will be described with reference to FIG. 6. Illustrated in FIG. 6 are experimental results of relative sensitivity plotted in relation to gas concentration when a gas sensor mountable to a microphone part of a cellular phone or mountable externally of the cellular phone is so sized that a 0.5 mm square-size detection film can exhibit a value of sensitivity of 1 to detectable gas. As will be seen from the figure, the conventional SnO₂ film (1 μm thickness) 12 (see FIG. 2 or 4) is sensitive to methylmercaptan, trimethylamine and acetone but is insufficiently sensitive to hydrogen. Even if the material of the sensor detection film is changed to WO₃ adapted for hydrogen, practically necessary sensitivity to 1000 ppm of hydrogen cannot be obtained with a 0.5 mm square film even with a catalytic film for dissociation of a hydrogen molecule into atomic state hydrogen optimized, so long as the film is produced through the conventional sputtering and oxidation process.

In contrast, with a WO₃ film (1 μm thickness) 13 (see FIG. 2 or 4) formed on a glass substrate heated to 500° C. through MOCVD process using a raw material of diketone complex, sensitivity up to 100 ppm of hydrogen can be obtained. Presumably, the present invention differs in optimum production method from Patent Document 1 (JP-A-2001-59160) in that WO₃ has a tendency toward columnar crystal, the MOCVD can minimize the diameter of columnar crystal to the most and as a result the surface area can be maximized. Thus, the CVD conventionally considered to be unsuitable is applied to WO₃ to find out the effect of sensor through teachings of the present invention for the first time, thereby ensuring that the function to analyze expiration containing hydrogen can be incorporated in the mobile radio terminal.

In connection with the second object of the invention, any of voiceprint in utterance of a user, password input by the user and authentication omissible procedure, either pre-registered by the user in advance or taken by the user before each expired-gas analysis, is introduced to the portable health check apparatus so as to provide a user for which privacy protection is indispensable with the personal authentication function and to provide a user for which privacy protection is unnecessary with the personal authentication omissible function, thus advantageously permitting all users to deal with the problem of privacy.

In connection with the third object of the invention, a call-up sound or call-up vibration of the cellular phone is generated at a time point set by the user or a doctor appointed by the user to enable the user to hook up the phone so as to have expiration collected, so that the user prone to forget the expiration analysis can advantageously be caused to carry out the expiration analysis periodically.

In connection with the fourth object of the invention, conducting an expiration analysis examination on the basis of data stored in a database of center server or a decision made by a doctor by taking advantage of the radio communication function of the cellular phone and displaying the results on the screen of the cellular phone can be materialized by a statistic process at the center server or by inquiring a doctor, with the result that an expiration analysis service in the case of an expert diagnosis being made for the sake of health care can be realized to advantage.

In connection with the fifth object of the invention, the cellular phone has the position detecting function and means for deciding in an emergency whether a user is staying near a hospital to which a doctor the user has contracted with belongs in order that a reservation contact is made to the doctor in charge when the user is staying near the hospital and the doctor in charges is in the hospital or for displaying a hospital near the user on the cellular phone screen and a reservation and user's disease history information are transmitted from the center server to nearby hospital when the user is staying near his or her family hospital but the doctor in charge is out or the user is staying remotely from the family hospital, whereby in case the user is a liver/kidney disease patient and monitored periodically under the direction of a diagnosis made by the doctor, the patient can be supported to advantage even when the patient being out happens to fall into bad condition and is required of an urgent close examination.

Other objects, features and advantages of the invention will become apparent from the following description of the embodiments of the invention taken in conjunction with the accompanying drawings.

### BRIEF DESCRIPTION OF THE DRAWINGS

FIG. 1 is a front view of an example of a portable health check apparatus provided with a gas sensor arranged internally of a mobile radio terminal according to an embodiment of the invention.

FIG. 2 is a plan view showing how a plurality of detection films are arranged in the gas sensor built in the portable health check apparatus in the FIG. 1 example.

FIG. 3 is a front view of another example of a portable health check apparatus provided with a gas sensor internally of a mobile radio terminal in the embodiment of the invention.

FIG. 4 is a plan view showing how an expiration collection window and a plurality of detection films are arranged in the gas sensor provided externally of the portable health check apparatus in the FIG. 3 example.

FIG. 5 is a block diagram showing a circuit construction of the portable health check apparatus according to the invention.

FIG. 6 is a graphic representation showing experimental results of relative sensitivity plotted in relation to gas concentration when a detection film of 0.5 mm square size exhibits a value of sensitivity of 1 to detectable gas.

FIG. 7 is a flowchart showing user authentication procedures in the portable health check apparatus according to the invention.

FIG. 8 is a diagram showing a model useful to explain an expiration analysis service method according to the invention.

FIG. 9 is a diagram showing a model useful to explain an expiration analysis service method cooperative with GPS position detection system according to the invention.

FIG. 10 is a diagram showing an example of a position information display screen according to the invention.

FIG. 11 is a diagram showing another example of the position information display screen according to the invention.

### DESCRIPTION OF THE EMBODIMENTS

### Embodiment 1

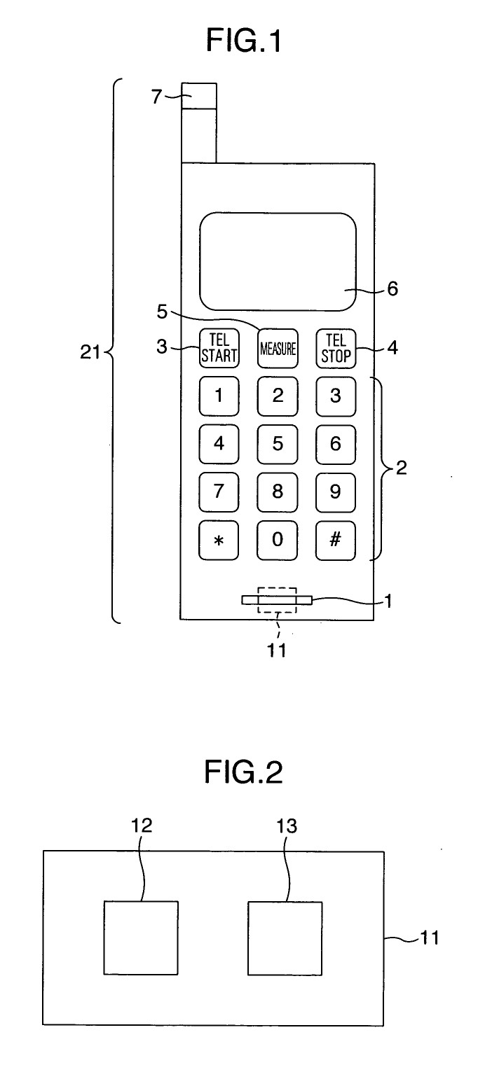

Referring to FIGS. 1 to 6, a first embodiment of this invention will be described.

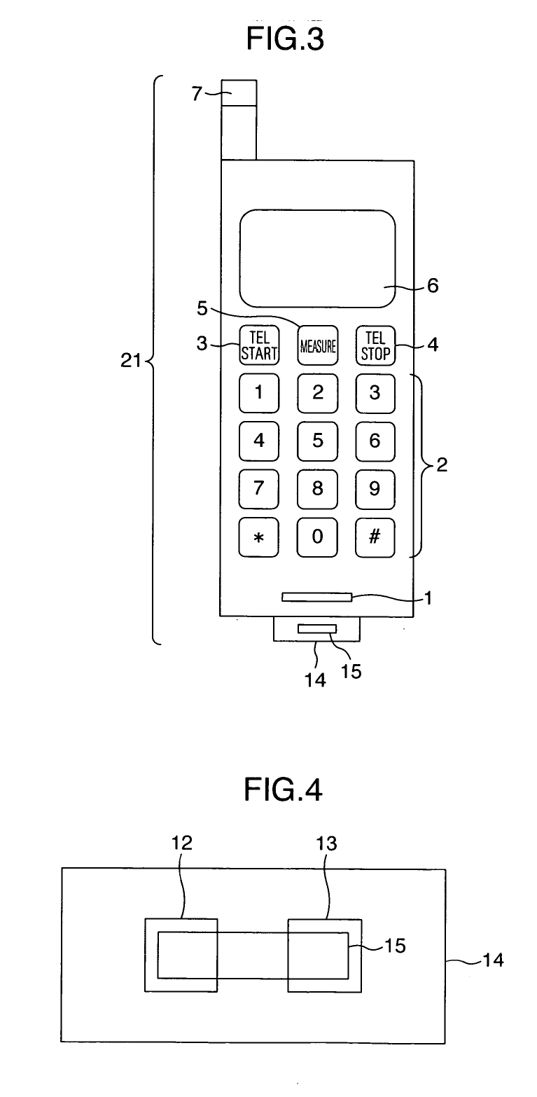

Illustrated in FIGS. 1 and 3 are front external appearance views of different examples of a portable health check apparatus according to the present invention. In the front, there are provided at least a microphone window 1, input ten keys generally designated at 2, a telephone start button 3, a telephone stop button 4, an expiration analysis start button 5, a display 6, an antenna 7 and a gas sensor 11 or 14. As shown in FIG. 1, the gas sensor 11 is disposed internally of the microphone window 1 of portable health check apparatus and as shown in FIG. 3, the gas sensor 14 is disposed externally of the portable health check apparatus. In case the gas sensor is disposed externally as shown in FIG. 3, connectors for connecting a charger to a cellular phone can conveniently be utilized and in this embodiment this configuration is used. Illustrated in plan view form in FIGS. 2 and 4 are disposition configurations of detection films in the gas sensors 11 and 14, respectively.

In the gas sensor 14 mounted to the portable health check apparatus externally thereof as shown in FIG. 4, the microphone window 1 for cellular phone cannot be used in common to the gas sensor and therefore an expiration collection window 15 is provided. In both types, a 0.5 mm square SnO₂ film (1 m film thickness) 12 and a 0.5 mm square WO₃ film (1 μm film thickness) 13 were used as detection films, respectively. The SnO₂ film (1 μm film thickness) 12 was deposited at room temperature through sputtering process and was annealed at 500° C. for 30 minutes in oxygen atmosphere. On the other hand, the WO₃ film (1 μm film thickness) was formed on a glass substrate heated to 500° C. through MOCVD process using a raw material of diketone complex, having its surface deposited with an catalytic film of Pd (15 nm film thickness). In the latter, annealing in the oxygen atmosphere was not particularly carried out after the MOCVD process.

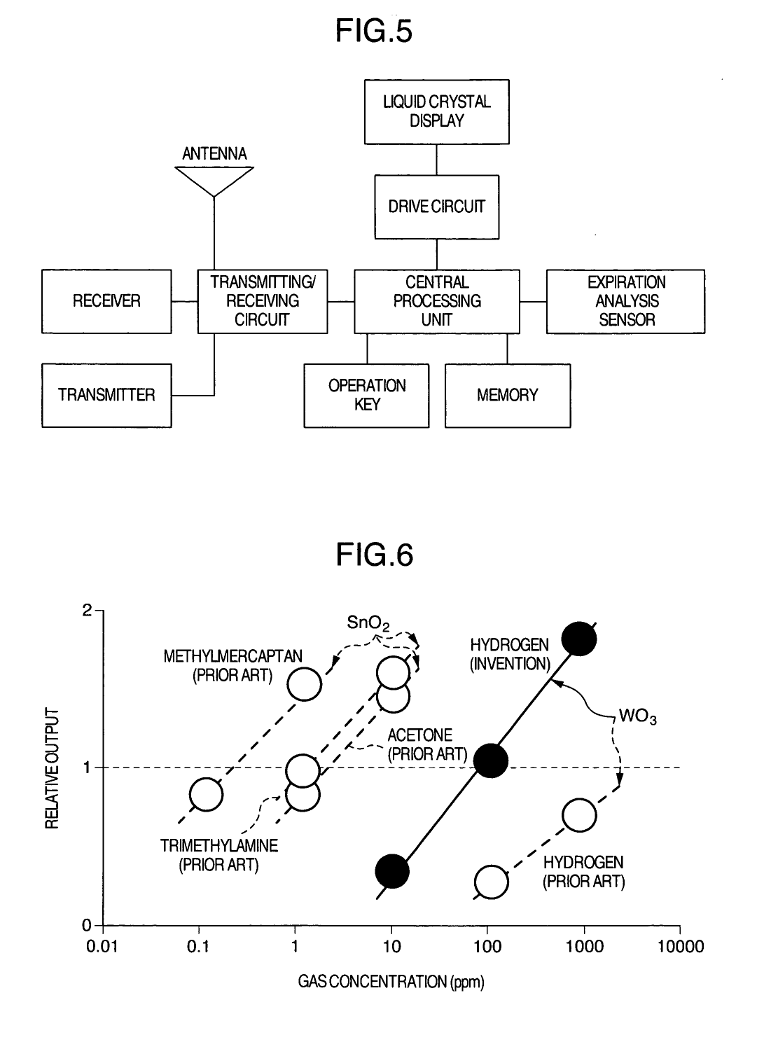

Referring now to FIG. 5, there is illustrated in block diagram form a circuit configuration of the portable health check apparatus in the present embodiment. A central processing unit for carrying out processes based on programs and data stored in a memory is connected with a liquid crystal display 6 via a driver circuit and results of operation/process by the central processing unit are displayed. On the other hand, a transmitting/receiving circuit is connected with an antenna, a transmitter and a receiver and a voice signal from the transmitter is converted into a predetermined transmission signal by means of the transmitting/receiving circuit and then transmitted through the antenna. A receiving signal from the antenna is converted into a voice signal by means of the transmitting/receiving circuit and then supplied to the receiver. According to the present embodiment, in addition to the above components of a usual cellular phone, the gas sensor made from the SnO₂ film 12 and adapted for methylmercaptan, trimethylamine and acetone and the gas sensor made from the WO₃ film 13 to act as a hydrogen sensor are provided so as to be connected to the central processing unit, thus forming the portable health check apparatus.

Values of sensitivity to methylmercaptan, trimethylamine, acetone and hydrogen were analyzed by using the portable health check apparatus explained in connection with FIGS. 1 to 5 to provide results as shown in FIG. 6. A practically necessary value of sensitivity to 1000 ppm hydrogen could not be obtained with the 0.5 mm square WO₃ film produced through the conventional sputtering process whereas a value of sensitivity up to 100 ppm could be obtained with the WO₃ film of the invention.

According to the invention, the WO₃ film formed through the MOCVD process is used for the hydrogen sensor and consequently, the portable health check apparatus having the expiration analysis gas sensor including the hydrogen sensor and mounted to the microphone part of the cellular phone or mounted externally of the cellular phone can be materialized to advantage.

In the present embodiment, only two of the SnO₂ film 12 and WO₃ film 13 are juxtaposed but the SnO₂ film fails to detect methylmercaptan, trimethylamine or acetone selectively and therefore, in an application for exactly separable measurement of these gases, a plurality of SnO₂ films 12, for instance, may obviously be prepared having thereon catalytic films suitable for detection of these gases, respectively, or alternatively metal oxide films having values of sensitivity to methylmercaptan, trimethylane and acetone gases different from that of the SnO₂ may be juxtaposed with the SnO₂ films, respectively.

### Embodiment 2

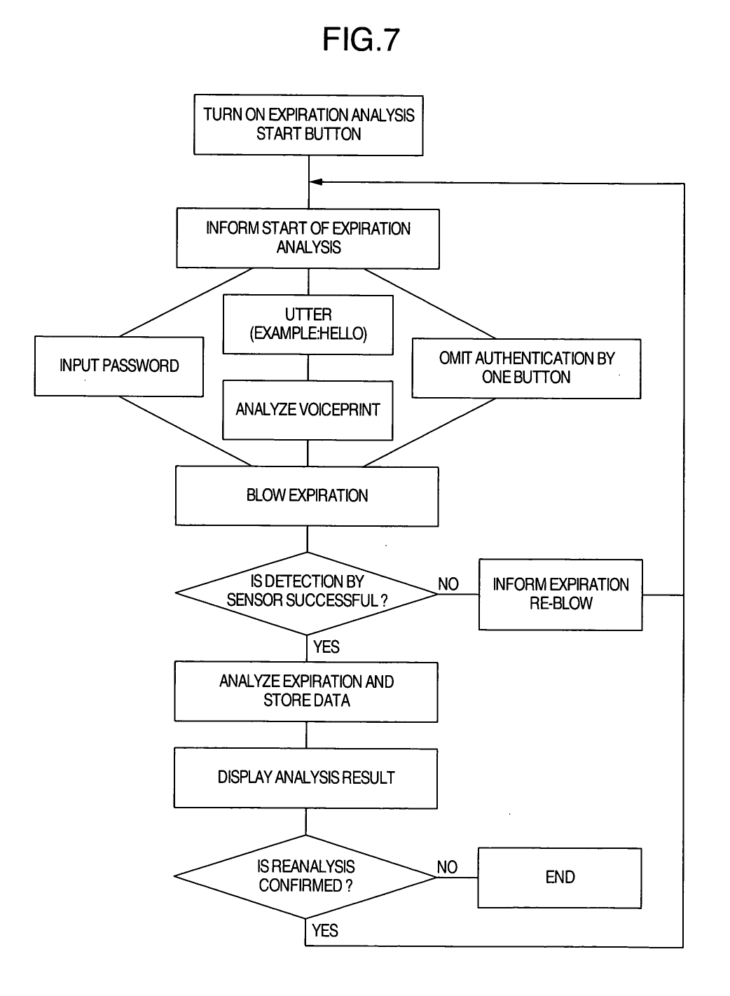

A second embodiment of the invention will be described by making reference to FIGS. 1, 3, 5 and 7. Illustrated in FIG. 7 is a flowchart showing procedures in authentication of a user of the portable health check appatatus. It is to be noted that normally, the cellular phone is always standby for reception and therefore the power supply is kept to be turned on. But if the procedures are started from the power supply turn-off state, the flowchart is started with truning on the main power supply of the cellular phone.

It is presupposed that the cellular phone is first placed in reception standby condition and then the user depresses the button 1 or 3 labeled as “measurement” to start expiration analysis measurement. Subsequently, the portable health check apparatus gives an announcement of expiration analysis start by voice uttering “Expiration analysis will now be started”. In this phase, giving a telephone call-up sound may be set in favor of a user who does not want others to know the measurement of expiration component in progress or generating vibration may be set in favor of a user staying in, for example, a public space and being prevented from uttering or of a user having difficulty in hearing.

Personal authentication has not hitherto been incorporated in the cellular phone having a built-in halitosis or alcohol sensor but in the present embodiment this is adopted in three ways as will be described below.

A first user authentication method utilizes a voiceprint accompanying utterance by a user such as “Hello”. The user has his or her voiceprint registered in advance in an authentication office connected through the Internet and each time that an expiration analysis is conducted, receives personal authentication based on the voiceprint through the Internet. This authentication utilizing Internet connection can be effected without consciousness of the user and advantageously, any procedural burden is not imposed on the user. To add, in the present embodiment, the well-known voice recognition and voice synthesis techniques necessary for voice registration/authentication are used.

In a second user authentication method, the use is requested to register in advance a password in the portable health check apparatus and is further requested to input the password each time that an expiration analysis proceeds. By virtue of omission of the authentication through the Internet, time of shift to the actual expiration analysis can be shortened but on the other hand, inputting of the password requires labor and time by such a slight amount that much load is not imposed on the user to complete authentication. In the present embodiment, a numeral of 4 figures is used as the password.

In a third user authentication method, the user is requested to either pre-register an authentication omissible procedure in advance or take the procedure before each expiration analysis to omit authentication. Originally, this is for convenience of a user who considers that authentication is unnecessary but it can comply with an instance in which a person other than the user attending as a guest utilizes the expiration analysis for trial. In the present embodiment, a method is adopted in which setting “Omit authentication by re-depressing the “start” button in FIG. 1 or 3” in advance is provided in a user menu and the user having done the setting can depress the “start” button each time before conducting expiration analysis to omit authentication.

When the result of the user personal authentication according to any of the above methods determines that the user of the portable health check apparatus has a right to utilize the apparatus, a voice “Please blow expiration” is announced to urge the user to blow. In this phase, setting of exhibiting a display in the form a character on the display 6 shown in FIG. 1 or 3 can be permitted in favor of a user who does not want others to know the measurement of expiration component in progress, a user being prevented from uttering in, for example, a public space or a user having difficulty in hearing or setting of making vibration can be permitted in favor of a user having bad sight.

When the sensor succeeds in detecting an expiration component after the user has blown expiration into the sensor part in a manner similar to talking on the usual cellular phone, data is stored in the memory shown in FIG. 5 and besides a result is displayed on the display. But in the event that the detection of expiration component is troubled, setting of urging the user to conduct re-measurement can be permitted by using voice, display or vibration.

Finally, setting for causing the user to confirm whether re-analysis is necessary can be permitted by using voice, display or vibration.

According to the present embodiment, the method for personal authentication based on any of voiceprint accompanying uttering “Hello” by the user of the portable health check apparatus, password input and authentication omissible registration can be incorporated in the portable health check apparatus and consequently, the portable health check apparatus respecting privacy without burdening the user can advantageously be realized.

### Embodiment 3

A third embodiment of the invention will be described hereunder.

In the present embodiment, the portable health check apparatus explained by making reference to embodiments 1 and 2 is incorporated with the timer function. A concrete timer utilization method can be exemplified, according to which a user per se of portable health check apparatus prone to forget an expiration analysis for which data collection is required daily at a constant or predetermined time or a person in charge of health care of the user participates in such setting that the user of the portable health check apparatus is urged to conduct the expiration analysis daily at the constant time through a call-up sound by telephone or vibration.

According to the present embodiment, setting of giving, at a predetermined time, a call-up sound or vibration for urging the user to utter and therefore, a portable health check apparatus capable of conducting an expiration analysis periodically can be realized to advantage.

### Embodiment 4

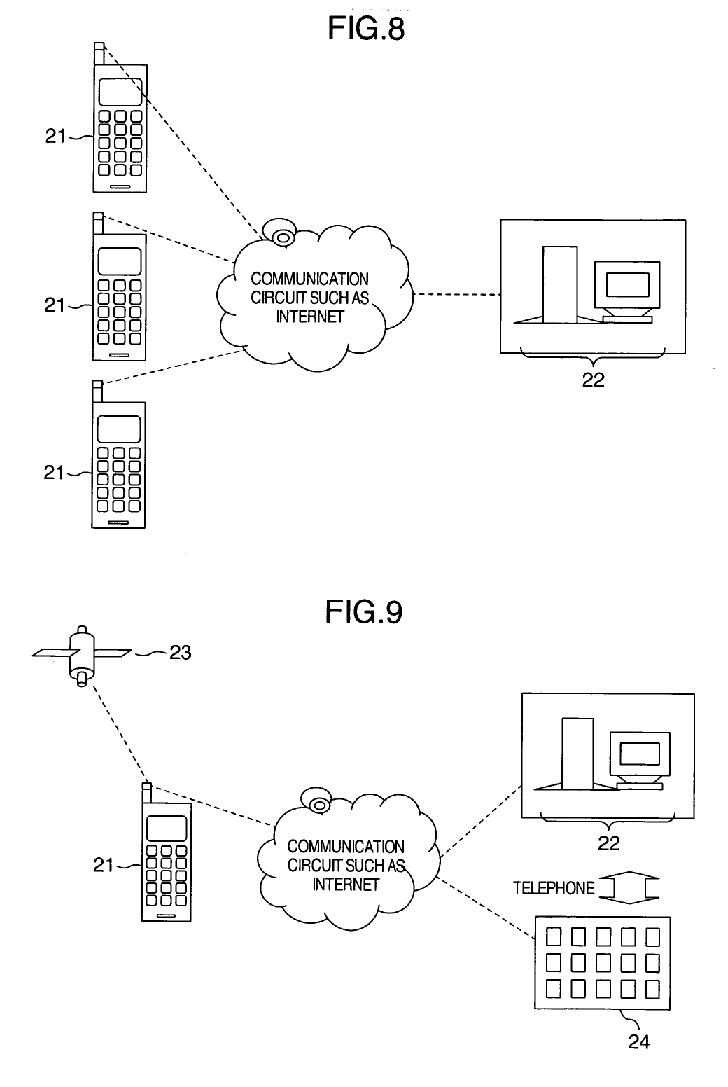

A fourth embodiment of the invention will be described by using FIG. 8.

Illustrated in FIG. 8 is a model showing an expiration analysis service method. A plurality of portable health check apparatuses 21 (see FIGS. 1 and 3) explained in connection with embodiments 1 to 3 are connected to a center server 22 through a communication circuit such as the Internet.

In the present embodiment, a system is structured in which detection information concerning an expiration component of a user of the portable health check apparatus 21 is received, via the communication circuit, by the center server which in turn analyzes or statistically processes the detection information, collates a result of the analysis or statistic process with advice information stored in a database and transmits advice information based on a result of the collation from the server to the user of the portable health check apparatus, thereby ensuring that an expiration analysis service given to the user of the portable health check apparatus can be practiced.

According to the present embodiment, in connection with, for example, a health care support for a patient suffering from a liver/kidney disease and living a usual life at home, an expiration analysis service can advantageously be provided which can enable an expert diagnosis for health care to be offered even when a decision cannot be made within the personal cellular phone.

### Embodiment 5

A fifth embodiment of the invention will be described with reference to FIGS. 9 to 11.

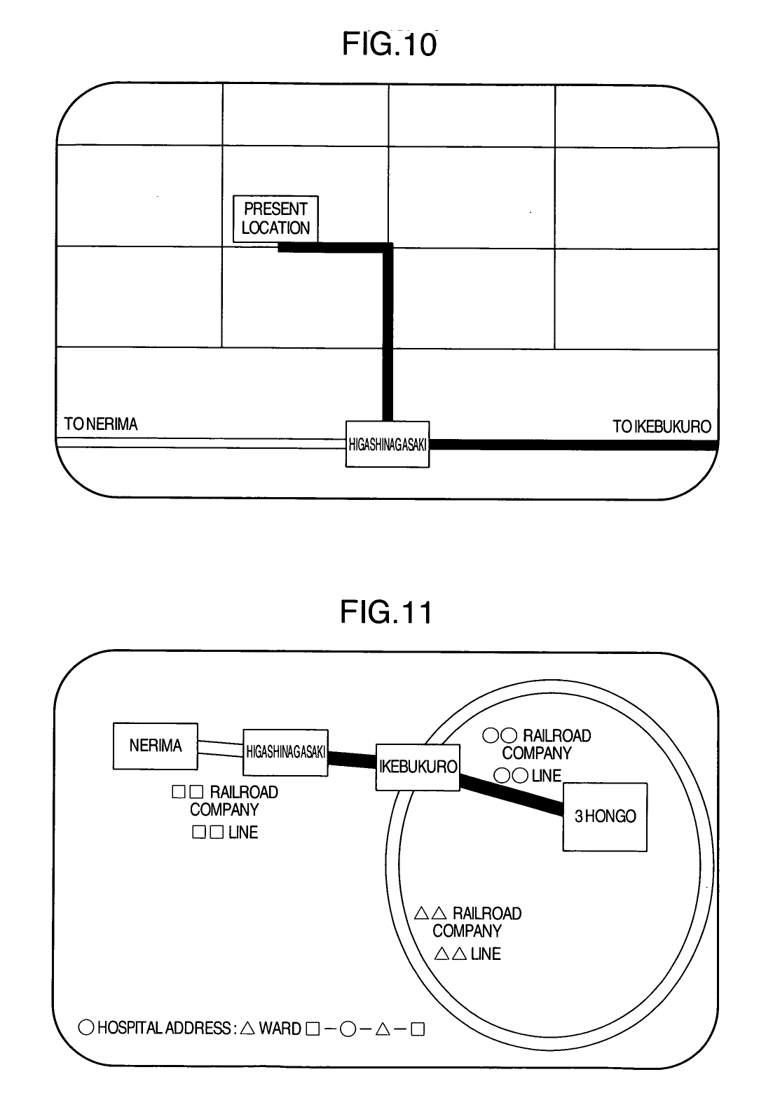

Illustrated in FIG. 9 is a model useful to explain an expiration analysis service method using the GPS (global positioning system). Examples of positioning information displayed on the display 6 (see FIGS. 1 and 3) of portable health check apparatus 21 are shown in FIGS. 10 and 11, respectively.

The portable health check apparatus 21 receives position detection basic information from a GPS satellite group 23. On the other hand, the center server 22 connected to the apparatus 21 via a communication network, for example, the Internet analyzes or statistically processes detection information concerning an expiration component exhaled by a user and collates a result of the analysis or statistic process with advice information stored in a database. In case the detection information detected by the sensor indicates emergency when transmitting advice information based on a result of the collation from the server to the portable health check apparatus of the user, the server informs a hospital 24 the user usually attends, automatically by telephone via the communication circuit, of the detection information and position information of the portable health check apparatus. Then, in the hospital receiving the detection information and position information through the Internet, a doctor in charge of the user confirms these pieces of informaiton. When the doctor in charge designates to an expiration analysis service company a hospital to which the user is to go by taking the emergency and the position information into consideration, the expiration analysis service company operates to cause a map (FIG. 10) indicative of the way to a nearby station of the user and an address of the hospital the user is to go to and traffic means (FIG. 11) to be displayed on the screen of the portable health check apparatus.

According to the present embodiment, a support means can be provided which is adapted to the case in which a patient suffering from liver/kidney disease and being monitored periodically through examination by a doctor happens to fall into bad condition while being out and an urgent close examination is needed.

It should be further understood by those skilled in the art that although the foregoing description has been made on embodiments of the invention, the invention is not limited thereto and various changes and modifications may be made without departing from the spirit of the invention and the scope of the appended claims.

1. A portable health check apparatus comprising:
a mobile radio terminal; and
a gas sensor mounted to said mobile radio terminal at least internally and externally thereof, said gas sensor including a hydrogen sensor,
wherein information concerning the health of human body to which a sense object of said gas sensor belongs is displayed on said mobile radio terminal on the basis of a result of sensing by said gas sensor.

2. A portable health check apparatus according to claim 1, wherein said sense result is processed inside said mobile radio terminal and the thus processed sense result is displayed.

3. A portable health check apparatus according to claim 1, wherein said sense result is processed in a center server connected to said mobile radio terminal via a communication circuit and said mobile radio terminal receives the thus processed sense result directly or indirectly from said center server to display it.

4. A portable health check apparatus according to claim 1, wherein said hydrogen sensor includes, as a hydrogen detectable material, tungsten oxide produced through a metalorganic chemical vapor deposition process.

5. A portable health check apparatus according to claim 1 further comprising means for functioning to authenticate a user utilizing a service relating to sensing by said gas sensor.

6. A portable health check apparatus according to claim 5, wherein said authentication is carried out on the basis of a voiceprint accompanying utterance by said user.

7. A portable health check apparatus according to claim 5, wherein said authentication is carried out on the basis of inputting of a password of said user.

8. A portable health check apparatus according to claim 5, wherein said authentication is so defined as to be omissible through an authentication omissible procedure, either pre-registered by the user in advance or taken by said user before each expiration analysis.

9. A portable health check apparatus according to claim 1 further comprising means for urging said user to utter at a predetermined time point.

10. A portable health check apparatus according to claim 9, wherein said urging means has the function to give at least one of call-up sound and vibration to said user.

11. An expiration analysis service method comprising the steps of:
receiving, at a center server from said portable health check apparatus via a communication circuit, detection information concerning an expiration component exhaled by a user of a portable health check apparatus and detected by said portable health check apparatus;
applying an information process to said detection information at said center server;
collating a result of said information process with a plurality of pieces of advice information stored in a database accessible by said center server; and
transmitting, from said center server, device information selected from said plurality of advice information pieces on the basis of a result of said collation directly or indirectly to said portable health check apparatus belonging to said user,
wherein said portable health check apparatus includes:
a mobile radio terminal; and
a gas sensor mounted to said mobile radio terminal at least internally and externally thereof, said gas sensor including a hydrogen sensor,
wherein information concerning the health of human body to which a sense object of said gas sensor belongs is displayed on said mobile radio terminal on the basis of a result of sensing by said gas sensor.

12. An expiration analysis service method according to claim 11 further comprising the step of distributing said portable health check apparatus to said user.

13. An expiration analysis service method according to claim 11, wherein said information process includes at least one of analytic and statistic processes applied to said detection information.

14. An expiration analysis service method according to claim 11, wherein said sense result is processed inside said mobile radio terminal and a processed sense result is displayed.

15. An expiration analysis service method according to claim 11, wherein said sense result is processed at said center server connected to said mobile radio terminal via the communication circuit and said mobile radio terminal receives a processed sense result directly or indirectly from said center server to display the received, processed sense result.

16. An expiration analysis service method according to claim 11, wherein said hydrogen sensor includes, as a hydrogen detectable material, tungsten oxide produced through a metalorganic chemical vapor deposition process.

17. An expiration analysis service method according to claim 11 further comprising functioning to authenticate a user utilizing a service relating to sensing by said gas sensor.

18. An expiration analysis service method according to claim 17, wherein said authentication is carried out on the basis of a voiceprint accompanying utterance by said user.

19. An expiration analysis service method according to claim 17, wherein said authentication is carried out on the basis of inputting of a password of by said user.

20. An expiration analysis service method according to claim 17, wherein said authentication is so defined as to be omissible through a authentication omissible procedure, either pre-registered by the user or taken by said user before each expiration analysis.

21. An expiration analysis service method according to claim 11, wherein said portable health check apparatus includes means for urging said user to utter at a predetermined time.

22. An expiration analysis service method according to claim 21, wherein said urging means has the function to give at least one of call-up sound and vibration to said user.

23. An expiration analysis service method according to claim 11 further comprising the steps of:
deciding whether information indicative of emergency is contained in said detection information;
when information indicative of emergency is determined as being contained in said detection information, informing a hospital registered in advance, from said center server through said communication circuit, of said detection information and position information concerning a location position of said portable health check apparatus;
confirming a doctor in charge of said utilizer in said hospital registered in advance and receiving information including said detection information and the position information concerning the location position of said portable health check apparatus;
causing said doctor in charge to confirm said detection information and the position information concerning the location position of said portable health check apparatus;
causing said doctor in charge to designate a hospital to which said user is to go; and
transmitting, to said health check apparatus, information relating to a location of the hospital to which said user is to go and displaying the information on the screen of said portable health check apparatus.

24. An expiration analysis service method according to claim 23, wherein said hospital registered in advance is a hospital said user attends usually.

25. An expiration analysis service method according to claim 23, wherein the information relating to a location of the hospital to which said user is to go includes at least an address of said hospital and its map.
